(19) United States
(12) Patent Application Publication
(10) Pub. No.: US 2004/0024178 A1
(43) Pub. Date: Feb. 5, 2004

(54) Bacterial signal peptidase inhibitors and uses thereof

(76) Inventors: Stephen Ashman, Harlow (GB); Michael T. Black, Collegeville, PA (US); Gordon Bruton, Harlow (GB); Alfred John Humphries, Harlow (GB); Keith James Millan Moore, Harlow (GB)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/256,850

(22) Filed: Sep. 27, 2002

Related U.S. Application Data
(63) Continuation of application No. 09/890,633.

(30) Foreign Application Priority Data
Feb. 3, 1999 (GB) 9902399.6

(51) Int. Cl. C07K 7/08; C07K 7/06

(52) U.S. Cl. 530/324; 530/326; 530/327; 530/328

(57) ABSTRACT

Substrates for bacterial signal peptidases and their use in assays to detect inhibitors of these enzymes.

This invention relates to compounds, processes for preparing them and their use as enzyme substrates.

Bacterial signal peptidases play a key role in protein secretion. The physiological function of these enzymes is to cleave off the signal sequence which targets the protein to the cytoplasmic membrane thereby ensuring release of mature protein from the outer surface of the membrane. In the absence of signal peptidase activity protein export ceases resulting in the accumulation of unprocessed protein and inhibition of cell growth. (Dalbey, Wickner, J Biol Chem 1985, 260(29) 15925). Consequently inhibitors of bacterial signal peptidases represent potentially important candidates for antibacterial action (Allsop et al., Biomed. Chem. Lett., 1995, 5 (5), 443).

Signal peptidases do not appear to recognise a specific amino acid sequence in native protein substrates. Nevertheless certain patterns have been discerned: the N-terminus of the signal sequence contains a hydrophobic membrane spanning helical domain followed by a more polar region which precedes the cleavage site. The cleavage site is usually preceded by the sequence AXA(A=small residue e.g. Ala; X=any amino acid; A=small residue e.g. Ala). (von Heijne, Eur. J. Biochem., 1986, 133, 17).

Several assay systems for assessing inhibition of processing by signal peptidases have been described. In the most commonly employed variant processing of either a peptide or protein substrate is measured using an HPLC based assay (Dev et al. J Biol Chem., 1990, 265, 20069). Inouye and coworkers have described a hybrid protein substrate (pro-Ompa-nuclease A) consisting of the signal sequence of E. coli Ompa (outer membrane protein A) fused to the mature portion of nuclease A from S. aureus (Chatterjee et al., J. Mol. Biol., 1995, 245, 311). Dierstein and Wickner (EMBO J. 1986, 5(2) 427) have described peptide substrates for E. coli leader peptidase based on the M13 procoat protein. A 23 amino acid peptide (ASVAVATLVPMLSFAAEGDDPAK) comprising the −15 to +8 residues of the M13 procoat protein was cleaved at a rate that was comparable to that of the parent but smaller fragments were cleaved much less efficiently. A related sequence AcWLVPNLLSFAAEGDDPANH₂ has also been described (Kuo et al., Arch. Biochem. Biophys., 1993, 303 (2), 274-278). Dev et al (J. Biol. Chem. 1990, 265(33), 20069) have reported the sequence FSASALAKI which corresponds to the −7 to +2 sequence of maltose binding protein. The shorter sequence ALAKI is the minimum required for processing but the rate of turnover is very slow. The related sequences WSASALAKI and AcWSASALAKI have also been described (Kuo et al., Arch. Biochem. Biophys., 1993, 303 (2), 274-278). This sequence has been exploited for the preparation of a fluorescent substrate for continous assay AcSASALAKI-AMC (AMC=aminomethylcoumarin).

The substrate FSASALAKI (Dev et al above) has been labelled with the fluorescence quench pair 3-nitrotyrosine and anthraniloyl to provide an internally quenched fluorescent substrate for E. coli leader peptidase (Zhong et al., Analytical Biochemistry 255, 66-73 (1998).

All the synthetic peptides described previously are inferior substrates for leader peptidase compared to protein substrates such as the fusion protein ProOmpa-NucleaseA (Chatterjee et al J. Mol. Biol. 1995, 245, 311).

No suitable substrates have been identified for other signal peptidases including Sps B of Staphylococcus aureus.

Novel compounds have now been identified which are substrates for bacterial signal peptidases and which are of use for configuring assays to detect inhibitors of these enzymes.

According to the present invention there is provided a compound of formula (I):

[A][A1]A2][A3]*[A4] (I)

where:

* is the cleavage site

[A] is selected from:

(i) 2 to 12 hydrophobic amino acid residues substituted at the N-terminus by C₁₋₅ alkanoyl or phenylC₁₋₄alkanoyl optionally substituted by C₁₋₄alkyl, C₁₋₄alkoxy or halogen; and

(ii) a hydrophobic acyl residue;

[A1] is 1 to 3 amino acid residues selected from A, F, G, I, L, M, N, S, T, V;

[A2] is 3 amino acid residues selected from those favoured in a beta- or helical turn;

[A3] is 3 amino acid residues X—B-Z, where X is selected from A, G, S, T, and V, B is any amino acid residue and Z is selected from A, G and S; and

[A4] is 2 to 8 amino acid residues chosen predominantly from those that are favoured in beta-turns and enhance water solubility:

and optionally wherein:

one amino acid in one of [A1] and [A2] is replaced by X1 comprising an amino acid bearing a marker group; and one amino acid in [A4] other than the residue immediately adjacent to [A3] is replaced by X2 comprising an amino acid bearing a marker group; such that X1 and X2 form a marker pair;

[A2] residues are selected from G, L, N, P, S and T;

X is selected from A, S and V;

B is selected from D, F, H, I, K, L, N, Q, R, V or Y; and

Z is selected from A and S.

In [A] (i) the hydrophobic amino acid residues are preferably selected from A, F, I, L, M, V and W, more preferably from A, F, I, L, M and V. Halogen is preferably chlorine. The N-terminal substituent is preferably acetyl.

In [A] (ii) the hydrophobic acyl residue is preferably selected from phenylC₁₋₁₂alkanoyl, biphenylC₁₋₁₂alkanoyl, phenoxyphenylC₁₋₁₂alkanoyl or C₅₋₁₆ alkanoyl, wherein the phenyl moieties are optionally substituted by C₁₋₈alkyl, C₁₋₈alkoxy or halogen. Halogen is preferably chlorine. [A] is more preferably C₅₋₁₆alkanoyl or para linked biphenyl C₁₋₁₂alkanoyl, more preferably C₈₋₁₂ alkanoyl, most preferably C₁₀ alkanoyl.

When [A] is a C₁₋₅alkanoyl- or phenylalkanoyl-end capped amino acid sequence [A1] is then preferably 1 amino acid chosen from G, L or N. When [A] is option (ii) [A1] is preferably 1 or 2 amino acids selected from F, I, L and V, most preferably 1 amino acid from F, I, L and V. [A1] is most preferably L.

[A2] is preferably 3 amino acid residues selected from A, F, G, I, L, N, P, S, T, V, preferably from G, L, N, P, S and T, more preferably from L, P, S and T. Most preferably [A2] is TPT or SLP. Where the compound of formula (I) bears the marker pair X1/X2, preferably P is not replaced by X1.

In [A3] X is preferably A, S or V, most preferably A, B is preferably a neutral or basic amino acid residue, preferably selected from F, H, I, K, L, N, Q, R, V or Y, more preferably H, K, R, N, L or Y, most preferably H, K or R and Z is preferably A or S, most preferably A.

In [A4] the first amino acid residue is preferably A, D, E, Q or S, most preferably A and the remaining amino acid residues are preferably selected from A, D, E, F, G, I, K, L, N, P, Q, R, S, T and V with not more than two of each of G, P and R and no more than 2 amino acid residues being selected from F, I, L and V. More preferably [A4] has 4 to 6⁻residues and these are selected from A, D, E, G, I, K, L, P, R, S, T and V. Most preferably [A4] has 6 residues selected from A, D, E, G, I, K, L, P, R, S, T and V with not more that one of each of G, P and R and not more than one residue selected from I, L and V.

For E. coli leader peptidase substrates [A4] preferably includes P and the remaining residues are preferably selected from A, D, E, G, L, R, S and T, preferably at least two residues being selected from D, E and R. More preferably the P is situated in the 2, 3 or 4 position from the cleavage site.

In a preferred aspect the compound of the invention is of formula (II):

[A]-a1-a2-a3-a4-a5-a6-a7-a8-a9-a10 (II)

where:

[A] is as defined in formula (I);

a1 is leu or X1;

and either

(i)

a2 is thr or X1;

a3 is pro;

a4 is thr or X1;

a5 is ala-(lys or arg or asn)-ala*-ala-;

a6 is ser or X2;

a7 is lys or X2;

a8 is ile or X2;

a9 is asp or X2; and

a10 is asp or X2;

or

(ii)

a2 is ser or X1;

a3 is leu or X1;

a4 is pro;

a5 is ala-(lys or arg or his)-ala*-ala-;

a6 is asp or X2;

a7 is leu or gly or X2;

a8 is pro;

a9 is arg or X2;

a10 is ser or X2.

Compounds of formula (II)(i) are particularly preferred substrates for Sps B of Staphylococcus aureus and compounds of formula (II) (ii) are particularly preferred substrates for E. coli leader peptidase.

The compounds of formula (I) contain a pair of marker groups, which straddle the cleavage site marked *. Cleavage separates the markers X1 and X2 and this change can be detected using techniques which reflect colocalisation of these markers. Detection may depend on an optical interaction between the two markers, or more generally, signal generation may be dependent upon their colocalisation in the substrate. Techniques based on optical interactions include fluorescent energy transfer (FQ), as described by Forster theory, and luminescence energy transfer (Selvin and Hearst, Proc. Nat. Acad. Sci. USA, 1994, 91, 10024). Assays based on changes in translational or rotational diffusion include fluorescence correlation spectroscopy (FCS) (Eigen and Rigler, Proc. Nat. Acad. Sci. USA, 1994, 91, 5740) and fluorescence polarisation (FP) (Levine et al., Anal Biochem., 1997, 247, 83). Radioactivity based assays include scintillation proximity assays and nitrocellulose filtration techniques. Surface adsorption techniques include immunoassays with either absorbance, fluorescence, chemiluminescence or time resolved fluorescence (TRF) detection (Wallac OY, Finland).

Thus, cleavage of the compound directly or indirectly results in the modulation of a signal, for example a radioactive, luminescent or fluorescent signal.

One marker group carries the signal generator or is capable of binding to a separate reporter system. The reporter system itself may carry the signal generator or may bind to a further signalling moiety. The other marker group performs the modulator function or is capable of binding a molecule such that the bound complex itself performs the modulator function.

Examples of signal generator groups include radioactive, luminescent (triplet state emission) and fluorescent (singlet state emission) labels.

Examples of marker groups capable of binding a separate reporter system include ligands for antibodies, enzymes and receptors. The antibody, enzyme or receptor reporter system is itself labelled or is capable of participating in an immnoassay. A suitable example of such a ligand is dinitrophenol which can be captured with anti-dinitrophenol antibody followed by a suitable immunoassay.

Examples of modulator groups include moieties which modulate the optical properties of the fluorescent or luminescent labels or of the substrate as a whole when both said label and moiety are attached covalently or non-covalently to the substrate. Upon proteolytic cleavage of the substrate, the optical properties of the label, or of the molecular entity as a whole, are modulated such that proteolytic activity can be monitored spectroscopically.

Examples of groups capable of effecting the modulator function or of binding a molecule to form a modulator complex include ligands for proteins, such as biotin ligands capable of binding streptavidin or avidin, or haptens for antibodies either in solution or immobilised. Biotin ligands include biotins optionally derivatised with suitable linker groups such as aminohexanoyl.

Examples of signal generator groups and other modulator groups which modulate the optical properties of a fluorescent signal generator include fluorophores (molecular families that exhibit absorption and fluorescence spectral ranges), such as coumarins, xanthenes (including rhodamines, rhodols and fluoresceins), fluorescamine derivatives, napthalenes, pyrenes, quinolines, resorufins, difluoroboradiazaindacenes, acridines, pyridyloxazoles, isoindols, dansyls, dabcyls, dabsyls, benzofuranyls, phthalimides, naphthalimides, and phthalic hydrazides (including luminol and isoluminol).

Examples of suitable label/modulator pairs include conventional fluorescence energy transfer or quenched fluorescence (FQ) donor/acceptor systems such as fluoresceins/rhodamines, fluoresceins/coumarins, 5-dimethylamino-1-naphthalenesulfonyl (DANSYL)/4-(4′-dimethylaminophenylazo)benzoic acid (DABCYL) or 5-(2-aminoethyl)aminonaphthalene-1-sulfonic acid (EDANS)/DABCYL whereby the absorption spectrum of the acceptor overlaps the emission spectrum of the donor such that changes in energy transfer are observed upon cleavage of the peptide according to Forster (1948) theory.

For detection using FQ, the X1/X2 marker group pair is a fluorophore pair, preferably chosen from amino acids bearing the combinations EDANS/DABCYL and fluoresceins/rhodamines. Most preferably the pair is 5-(and/or 6)-carbonylfluorescein with 5-(and/or 6)-carbonyltetramethylrhodamine.

Other examples of labels include lanthanide ions (typically terbium and europium) as luminescent donors for lanthanide resonance energy transfer to fluorescent or chromophoric acceptors (e.g exemplified by homogeneous time-resolved fluorescence (HTRF) technology or lanthanide chelate excitation (LANCE) technology). Spin-coupled quenching of a lanthanide donor is also possible with a nitroxide radical acceptor, typically a piperidinyloxy or pyrrolidinyloxy radical. (See M. V. Rogers (1997) DDT 2(4) 156).

Where the modulator group is a moiety which modulates the optical properties of the substrate as a whole upon proteolytic cleavage of the substrate, examples of suitable label/modulator pairs include a fluorescent label and a ligand for a protein, such as a biotin ligand capable of binding streptavidin or avidin. Changes in the rotational diffusion of the peptide resulting from cleavage can be monitored by observing changes in fluorescence polarisation (FP). Alternatively fluorescence correlation spectroscopy (FCS) can be used to mointor changes in translational diffusion.

Thus, for detection using FP or FCS, the X1/X2 marker groups are chosen from amino acids bearing a fluorophore, as defined above, combined with an amino acid bearing a protein ligand such as biotin derivatives, or haptens such as difluoroboradiazaindacenes, dansyls, dinitrophenols, fluorosceins, rhodamines and naphthalimides. The X1/X2 marker group pair is preferably a fluorophore/biotin ligand combination. Most preferably the pair is 5-(and/or 6)-carbonylfluorescein with aminohexanoyl linked biotin (biotin-X).

In a preferred aspect the the marker group pair provides a fluorescence-quench (FQ), a fluorescence-polarisation (FP) or a fluorescence correlation spectroscopy (FCS) assay.

X1 and X2 are preferably fluorophore and protein ligand derivatives of amino acids with side chains readily capable of chemical modification such as lysine, ornithine, cysteine, homocysteine, serine, homoserine and tyrosine.

In a preferred aspect X1 and X2 are or comprise modified lysine groups of the formula:

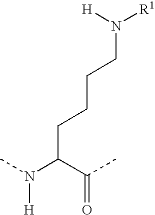

wherein R¹ is selected from suitable marker groups that are attached directly, or indirectly via a linking moeity, to the lysine, such that X1 and X2 together form a marker group pair as above described.

When X2 replaces the last amino acid of [A4] or when a10 in formula (II) is X2 it may alternatively be a tetrapeptide asp-B¹—Y-Z bearing the marker group. B¹, Y and Z¹ may be chosen from asp, glu, lys, arg, ser, ala or gly. B¹ is preferably gly, Y preferably carries the marker group as a modified lysine and Z¹ is preferably asp.

In one preferred aspect, in compounds of formula (II)(i) X1 is at a1 or a4 and X2 is at a8 or a10, more preferably a8.

In a second preferred aspect, in compounds of formula (II)(ii) X1 is at a1 or a3 and X2 is at a6, a7 or a9.

The compounds of formula (I) may be prepared by any appropriate conventional method of peptide synthesis. This includes strategies based on, for example, the Fmoc- and Boc- versions of solid phase synthesis and including sequential and fragment variations, or combinations thereof, for the chain assembly. Also are included the many different approaches for the chemical synthesis of peptides by the solution method, again utilising sequential or fragment assemblies, or combinations thereof. Other synthetic approaches can also be considered, such as those based on enzymatic coupling, etc. To those skilled in the art it will be realised that for the synthesis of peptides there are many variations possible, for example starting with different protecting groups, resins and linkers, coupling reagents, solvents, deblocking reagents, etc. Examples of such processes can be found in textbooks, including, for example, ‘Solid Phase Synthesis by J M Stewart and J D Young’, San Francisco, Freeman, 1969; ‘The Chemical Synthesis of Peptides’, J Jones, Clarendon Press, Oxford, 1991; ‘Principles of Peptide Synthesis’, M Bodanszky, Springer-Verlag, NY, NY, 1984; ‘Solid Phase Peptide Synthesis’, E Atherton and R C Sheppard, IRL Press, Oxford University Press, Oxford, 1989. More modern approaches are presented in the well known series of Proceedings from recent symposia, including, ‘Innovations and Perspectives in Solid Phase Synthesis’, Ed R Epton, and those conferences arranged by the European and American Peptide Societies and published under the title, ‘Peptides’.

Introduction of the group [A] is carried out by routine methods of N-terminal acylation.

Coupling of the marker groups to form X1/X2 is accomplished by conventional methods, for example by the addition under basic conditions of either an activated ester (e.g. succinimidyl), a mixed anhydride (e.g. ethoxycarbonyl), an acid chloride, a maleimide, an isocyanide or an isothiocyanide derivative of the marker group to the base substrate (resin bound or in solution) in which a single lysine, ornithine, serine or homoserine residue bears an unprotected primary amine or hydroxyl in the side chain. Alternatively, the base substrate (resin bound or in solution) in which a single cysteine or homocysteine residue remains unprotected is reacted with either a primary alkyl halide or maleimide derivative of the marker/reporter group under basic conditions. Alternatively, optionally protected fragments containing the required marker groups may be prepared and then assembled using standard coupling conditions.

Compounds of formula (I) are useful as substrates for bacterial signal peptidases, in particular S. aureus SpsB and E. coli leader peptidase, and are therefore useful in assay systems for testing for signal peptidase inhibitors.

According to a further aspect of the invention there is provided an assay system for testing for bacterial signal peptidase inhibitors which comprises contacting a bacterial signal peptidase and a compound of formula (I) with a test compound and measuring inhibition of the cleavage of the compound of formula (I) by the peptidase.

Suitable signal peptidases include S. aureus SpsB (Cregg K. M and Black M. T., J. Bacteriol. 1996, 5712) and E. coli leader peptidase (Wolfe, J. Biol Chem. 1983, 258(19) 12073). The peptidase may be provided in soluble form containing an N-terminal deletion (e.g. Tschantz, Biochemistry, 1995, 34,3935) or the peptidase may include a mutation to aid stability and/or purification. In a preferred aspect the gene encoding the N terminus of E. coli leader peptidase is mutated to remove the internal cleavage site (Ala-38 to Tyr and Ala-40 to Thr). A suitable mutation is indicated below:

| | |
|---|---|
| LPase | 5′-GTCAGGCAGCGGCGCAGGCGGCTCG-3′ |
| | \|\|\|\|\|\|\|\|\|\|\| \|\|\| \|\|\|\|\|\|\| |
| pLEX3 | 5′-GTCAGGCAGCGtatCAGaCGGCTCG-3′ |

(LPase=E. coli leader peptidase pLEX3=novel mutated gene)

The invention also extends to such novel mutated protein, processes for preparing it and its use in an assay system for testing for bacterial signal peptidase inhibitors as well as the novel mutated gene, vectors containing it and host cells transformed with such vectors.

The peptidases may be prepared by purification from the organism or conventional recombinant expression as described in, for example, Cregg K. M and Black M. T., J. Bacteriol. 1996, 5712 and Dalbey et al., Protein Science, 1997, 1129.

Generally the rate of cleavage in the absence of test compound will be known, as will the extent of cleavage at given time points. The assay may test for inhibition of cleavage at specified time points or of the rate of cleavage.

Substrate cleavage may be carried out either in solution or utilising a solid support.

The test compound may be pre-incubated with the appropriate signal peptidase enzyme prior to the addition of the substrate, or alternatively the substrate may be added directly. Final concentrations of enzyme and substrate are calculated so as to achieve a suitable rate of processing for carrying out the assay. The reaction may be stopped, for example by addition of methanol or trifluoroacetic acid, and the products analysed using any conventional system.

For example, reverse phase HPLC with UV detection can be used (see, for example, Kuo, D, et al., Biochemistry, 8347, 1994 and Allsop et al., Bioorganic and Med. Chem. Lett, 443, 1995). The activity of test compounds can be expressed as the % reduction in enzyme activity at given concentrations. In the HPLC asay this is calculated as the reduction in product peak area compared to the control. Where the compound of formula (I) contains a marker pair, methanol or an exogenous binding protein may alternatively be used to stop the reaction and the products analysed using any conventional system appropriate to the choice of marker groups utilised.

Radioactive methods include the use of a biotin/radiolabel pair. The substrate is captured onto streptavidin coated flashplates, streptavidin-coated scintillation proximity assay beads or by conventional filtration (e.g nitrocellulose) techniques. Detection of the radiolabel may be carried out by way of scintillation counting.

Antibody-based peptide detection methods include the use of a ligand/ligand pair e.g. biotin/dinitrophenol label. Following capture via one ligand e.g onto streptavidin coated plates, detection of the immobilised substrate is carried out using antibodies to the other ligand e.g antiDNP antibodies followed by an immunoassay such as enzyme linked immunosorbent assay (ELISA), dissociation enhanced time resolved fluorescence (DELFIA technology, Wallac OY), immunosorbent luminescence chemiluminescence or fluorescence detection.

Optical methods measuring changes in energy transfer can be carried out either macroscopically via total fluorescence intensity using a fluorimeter or by signal processing of photon emissions from individual fluorescence molecules via fluorescence correlation spectroscopy (FCS) using algorithms developed e.g. by Evotec Biosystems GmbH. Similar algorithms can be applied to determination of proteolysis rates using FCS via changes in the molecular brightness and particle number of dual and/or indirectly labelled peptide substrates.

Where the modulator group is a moiety which modulates the optical properties of the substrate as a whole upon proteolytic cleavage of the substrate, changes in fluorescence polarisation (FP) as a result of cleavage of e.g a dual biotinylated and fluorescent labelled peptide, either without or most preferably with the addition of e.g streptavidin or avidin, can be used to monitor protease activity. Changes in diffusion time of this fluorescently labelled peptide substrate as a result of proteolysis, either with or without the addition of streptavidin or avidin, can also be monitored by translational FCS. Fluorescence polarisation may be measured e.g. on a fluorescence polarisation platereader.

The invention also extends to compounds identified by the assay system of the invention and to pharmaceutical compositions containing them, their use as pharmaceuticals, in particular as antibacterial agents and methods of treatment of bacterial infection comprising administering to the sufferer a therapeutically effective amount of the compound so identified.

Another aspect of the invention is therefore a pharmaceutical composition comprising a compound identified by the invention and a pharmaceutically acceptable carrier.

The invention further relates to the use of a compound identified by the invention in the manufacture of a medicament for the treatment of bacterial infection.

The composition may be formulated for administration by any route, such as oral, topical or parenteral. The compositions may be in the form of tablets, capsules, powders, granules, lozenges, creams or liquid preparations, such as oral or sterile parenteral solutions or suspensions.

The topical formulations of the present invention may be presented as, for instance, ointments, creans or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Tablets and capsules for oral administration may be in unit dose presentation form, and may contain conventional excipients such as binding agents, for example syrup, acacia, gelatin, sorbitol, tragacanth, or polyvinylpyrollidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Suppositories will contain conventional suppository bases, e.g. cocoa-butter or other glyceride.

For parenteral administration, fluid unit dosage forms are prepared utilizing the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilised by exposure to ethylene oxide before suspending in the sterile vehicle. Advantageously, a surfactant or wetting agent is included in the composition to facilitate uniform distribution of the compound.

For administration to human patients, it is expected that the daily dosage level of the active agent will be from 0.01 to 50 mg/kg, typically around 1 mg/kg. The physician in any event will determine the actual dosage which will be most suitable for an individual patient and will vary with the age, weight and response of the particular patient. The above dosages are exemplary of the average case. There can, of course, be individual instances where higher or lower dosage ranges are merited, and such are within the scope of this invention.

With the indicated dose range, no adverse toxicological effects are indicated with the therapeutic compounds of the invention which would preclude their administration to suitable patients.

### EXAMPLES

### Example 1

Ac-Leu-Leu-Leu-Thr-Asn-Thr-Ala-Lys-Ala-Ala-Ser-Lys-Ile-Asp-Asp-OH (E1)

Compound E1 was assembled by continuous flow chemistry on a Millipore 9050 Peptide Synthesiser starting with 0.54 g, 0.0760 mmol, of Fmoc-Asp(OBu^{t})-OPEG-PS resin (PerSeptive Biosystems, 0.14 mmole/g). The coupling cycle consisted of the following stages: (1) 20% piperidine in DMF, 1 min; (2) DMF, 3 min; (3) 20% piperidine in DMF, 1 min; (4) DMF, 3 min; (5) 20% piperidine in DMF, completion of deprotection of the Fmoc group, 5 min; (6) DMF wash, 7 min; (7) coupling during a 1 hr recycle with the appropriate activated Fmoc-amino acid derivative; (8) DMF wash, 4 min; (9) double coupling by repeating step (7); and (10) DMF wash, 4 min. The activated amino acid derivatives were derived by treating a mixture of the appropriate Fmoc-amino acid derivative (0.8 mmole) containing equivalent amounts of TBTU and 1-hydroxybenzotriazole (HOBt) (Novabiochem) with a solution of diisopropylethylamine (DIPEA, 2 equivalents) in DMF.

After twelve coupling cycles, the resultant intermediate peptide resin, Fmoc-Leu- Thr(Bu^{t})-Asn(Trt)-Thr(Bu^{t})-Ala-Lys(Boc)-Ala-Ala-Ser(Bu^{t})-Lys(Boc)-Ile-Asp(OBu^{t})-Asp(OBu^{t})-PEG-PS resin was submitted to a further piperidine deprotection utilising steps (1) to (6) to give, H-Leu-Thr(Bu^{t})-Asn(Trt)-Thr(Bu^{t})-Ala-Lys(Boc)-Ala-Ala-Ser(Bu^{t})-Lys(Boc)-Ile-Asp(OBu^{t})-Asp(OBu^{t})-PEG-PS resin. Approximately one-half of this intermediate peptide resin was removed and the peptide assembly was continued on the remaining half, utilising two more coupling couples and an Fmoc-deprotection cycle.

The resulting H-Leu-Leu-Leu-Thr(Bu^{t})-Asn(Trt)-Thr(Bu^{t})-Ala-Lys(Boc)-Ala-Ala-Ser(Bu^{t})-Lys(Boc)-Ile-Asp(OBu^{t})-Asp(OBu^{t})-PEG-PS resin was transferred to a manual peptide synthesiser operating on the bubbler principle, and acetylated with a solution of acetic anhydride, 0.25 ml, in a small amount of DMF. Progress was monitored by the qualitative ninhydrin test.

After washing with DMF, methanol and ether, the dried Ac-Leu-Leu-Leu-Thr(Bu^{t})-Asn(Trt)-Thr(Bu^{t})-Ala-Lys(Boc)-Ala-Ala-Ser(Bu^{t})-Lys(Boc)-Ile-Asp(OBu^{t})-Asp(OBu^{t})-PEG-PS resin weighed 0.4379 g. The latter peptide resin was treated for 3 hr with 8 ml of a solution containing TFA, 15.2 ml, triisopropylsilane (TIS), 0.4 ml and water, 0.4 ml, with occasional swirling. The deblocked and cleaved peptide product was isolated by filtration of the TFA solution from the reaction mixture and rinsing the residual resin four times with fresh TFA. The combined TFA extracts were concentrated and the peptide precipitated by addition of a large excess of dry ether and cooling the mixture briefly in an acetone-dry ice bath. The solid peptide was collected by centrifugation and the pellet washed by vortexing in fresh ether followed by centrifugation. After a total of three washes with ether the product was dried to give 99.3 mg of crude peptide.

An aliquot of the crude peptide (25.2 mg), dissolved in about 1 ml of aqueous acetic acid and centrifuged to remove a little undissolved solid, was purified by hplc (Hypersil BDS C8, 21×250 mm, 6 ml/min, detection at 220 and 256 nm) with the gradient set at 5% B (time=0 min), 5% B (10 min), 95% B (130 min) (gradient name 5S2H95), where A was water, acetonitrile, TFA (1978, 20, 2) and B was acetonitrile, water, TFA (90, 10, 0.1). The purified product eluted at about 63.5 min and appropriate fractions were pooled and lyophilised to leave 3.9 mg of E1. Fraction selection was made on the basis of product purity and no attempt was made to optimise product recovery. The product eluted at 22 min with the gradient set at 5% B (0 min), 5% B (6 min), 95% B (36 min) (gradient 5N95D) and at 12.5 min with the gradient set at 30%B (Ornin), 30% B (6 min), 55% B (56 min) (gradient name 30S55D) on analysis by hplc (Hypersil BDS C8, 4.6×250 mm, 1 ml/min, detection at 220 and 256 nm). MS (ES) m/e 808.8 [M+2]²⁺. Amino acid analysis, acceptable ratios.

### Example 2

Decanoyl-Leu-Thr-Asn-Thr-Ala-Lys-Ala-Ala-Ser-Lys-Ile-Asp-Asp-OH (E2)

Compound E2 was produced similarly to Example 1. The common intermediate peptide resin, H-Leu-Thr(Bu^{t})-Asn(Trt)-Thr(Bu^{t})-Ala-Lys(Boc)-Ala-Ala-Ser(Bu^{t})-Lys(Boc)-Ile-Asp(OBu^{t})-Asp(OBu^{t})-PEG-PS resin was acylated in a manual bubbler with a solution of decanoic anhydride, 0.4 g, in a small amount of DMF for 30 min. After washing and drying, as given for Example 1, the decanoyl-Leu-Thr(Bu^{t})-Asn(Trt)-Thr(Bu^{t})-Ala-Lys(Boc)-Ala-Ala-Ser(Bu^{t})-Lys(Boc)-Ile-Asp(OBu^{t})-Asp(OBu^{t})-PEG-PS resin weighed 0.2998 g. Treatment of the latter peptide resin, as described for Example 1, with 8 ml of a solution of TIS and water in TFA gave 75.9 mg of crude peptide product. Preparative hplc of a centrifuged solution of 21.8 mg of the latter crude peptide in 1.4 ml of aqueous acetic acid, utilising the same method as described for Example 1, produced 3.14 mg of purified E2, with the product eluting at 79 min. On analytical hplc of E2, with the same system as given for Example 1, the product eluted at 26 min with the gradient 5N95D and at 35 min with the gradient 30S55D. MS (ES) m/e 751.8 [M+2]²⁺. Amino acid analysis, acceptable ratios.

### Example 3

Decanoyl-Leu-Thr-Asn-Thr-Ala-Lys-Ala-Glu-Ser-Lys-Ile-Asp-Asp-OH (E3)

(i) Assembly of Fmoc-Leu-Thr(Bu^{t})-Asn(Trt)-Thr(Bu^{t})-Ala-Lys(Boc)-Ala-Glu(OBu^{t})-Ser(Bu^{t})-Lys(Boc)-Ile-Asp(OBu^{t})-Asp(OBu^{t})-PS resin

One well of an ACT396 synthesiser was charged with 30 mg (0.0228 mmole) of Fmoc-Asp(OBu^{t})-Wang resin (Novabiochem, 0.76 mmol/g). Programmed peptide assembly operations were conducted using DMF as system fluid, drain times of 4 or 6 mins and washes with 1 ml of DMF for 30 sec at 300 rpm. The general assembly protocol consisted of (1) DMF, wash; (2) removal of Fmoc protection by two treatments with 20% piperidine in DMF (1 ml) for 5 min followed by a third treatment for 10 min; (3) DMF, 6 washes; (4) transfer or dispense 0.35 ml (0.175 mmol, 7.6 equiv) of the relevant Fmoc-amino acid derivative or Fmoc-amino acid derivatives (0.5M in DMF) and mix, 30 sec; (5) transfer 0.35 ml of a solution of HBTU (22.76 g) and HOBt (9.18 g) in 120 ml DMF (7.6 equiv of each reagent) and mix, 30 sec; (6) transfer 0.35 ml of a solution of DIPEA (34.8 ml) in DMF (165.2 ml) (15.2 equiv) and mix, 5 min; (7) flush pipetting arm and then couple for a further 60 min using steps of mix, 5 min, wait, 5 min, followed by a 6 min drain; (8) DMF, wash; (9) double coupling by repeating steps (4) to (7); (10) DMF, 4 washes; (11) treatment with 1 ml of a solution of acetic anhydride (20 ml) in DMF (200 ml) to cap any unreacted amino functions, mix, 5 min; (12) DMF, 5 washes; the final assembled resin bound peptides were harvested, washed with methanol and dried. In this way the intermediate peptide resins for Examples 4 to 18 were also constructed in parallel.

(ii) Completion of Synthesis and Purification

The resulting Fmoc-Leu-Thr(Bu^{t})-Asn(Trt)-Thr(Bu^{t})-Ala-Lys(Boc)-Ala-Glu(OBu^{t})-Ser(Bu^{t})-Lys(Boc)-Ile-Asp(OBu^{t})-Asp(OBu^{t})-PS resin was transferred to a manual bubbler and the Fmoc protection removed using (1) DMF, 5 washes, each 1 min; (2) two treatments with 20% piperidine in DMF for 5 min followed by a third treatment for 10 min; (3) DMF, 6 washes. The resultant H-Leu-Thr(Bu^{t})-Asn(Trt)-Thr(Bu^{t})-Ala-Lys(Boc)-Ala-Glu(OBu^{t})-Ser(Bu^{t})-Lys(Boc)-Ile-Asp(OBu^{t})-Asp(OBu^{t})-PEG-PS resin was acylated with decanoic anhydride to give decanoyl-Leu-Thr(Bu^{t})-Asn(Trt)-Thr(Bu^{t})-Ala-Lys(Boc)-Ala-Glu(OBu^{t})-Ser(Bu^{t})-Lys(Boc)-Ile-Asp(OBu^{t})-Asp(OBu^{t})-PEG-PS resin, as described for Example 2. This was treated with TFA-TIS-water, similar to that described for Example 1, except that the TFA extracts of the product were combined and evaporated to dryness then re-evaporated from fresh TFA two times and the residue was washed with small amounts of ether to produce, after drying, 37.1 mg of crude E3.

A centrifuged extract of the crude peptide in aqueous acetic acid was purified, in two portions, by hplc (Hypersil BDS C8, 10×250 mm, 4 ml/min, detection at 220 and 256 nm). The first half of the stock solution was submitted to the gradient set at 25% B (time=0 min), 25% B (10 min), 60% B (57 min), 95% (62 min) (gradient name 25S60P), where A was water, acetonitrile, TFA (1978, 20, 2) and B was acetonitrile, water, TFA (90, 10, 0.1) when the product eluted at about 36 min. The second half of the stock solution was purified with the gradient set at 25% B (time=0 min), 25% B (10 min), 55% B (57 min), 95% (62 min) (gradient name 25S55), and the product eluted at about 39 min. Appropriate fractions were combined and lyophilised to give 7.78 mg of purified E3. The purified product eluted at about 26 min with the gradient 5N95D, as described for Example 1. MS (ES) m/e 780.6 [M+2]²⁺. Amino acid analysis, acceptable ratios.

### Example 4

Decanoyl-Leu-Thr-Pro-Thr-Ala-Lys-Ala-Ala-Ser-Lys-Ile-Asp-Asp-OH (E4)

The intermediate peptide resin, Fmoc-Leu-Thr(Bu^{t})-Pro-Thr(Bu^{t})-Ala-Lys(Boc)-Ala-Ala-Ser(Bu^{t})-Lys(Boc)-Ile-Asp(OBu^{t})-Asp(OBu^{t})-PS resin, obtained from the ACT396 synthesis as described under Example 3, was converted to decanoyl-Leu-Thr(Bu^{t})-Pro-Thr(Bu^{t})-Ala-Lys(Boc)-Ala-Ala-Ser(Bu^{t})-Lys(Boc)-Ile-Asp(OBu^{t})-Asp(OBu^{t})-PS resin and then cleaved and deblocked as described for Example 3. The crude product, in aqueous acetic acid, was purified as described for Example 3 with the gradient 25S55 to give 10.32 mg of Example 4, when the product eluted at about 39.5 min. On analytical hplc the purified product eluted at about 36 min with the gradient 30S55D, as described for Example 1. MS (ES) m/e 743.1 [M+2]²⁺. Amino acid analysis, acceptable ratios.

### Example 5

Ac-Leu-Leu-Leu-Thr-Pro-Thr-Ala-Lys-Ala-Ala-Ser-Lys-Ile-Asp-Asp-OH (E5)

The intermediate peptide resin, Fmoc-Leu-Thr(Bu^{t})-Pro-Thr(Bu^{t})-Ala-Lys(Boc)-Ala-Ala-Ser(Bu^{t})-Lys(Boc)-Ile-Asp(OBu^{t})-Asp(OBu^{t})-PS resin, obtained from the ACT396 synthesis as described under Example 3, was transferred to a manual bubbler and converted to H-Leu-Leu-Leu-Thr(Bu^{t})-Pro-Thr(Bu^{t})-Ala-Lys(Boc)-Ala-Ala-Ser(Bu^{t})-Lys(Boc)-Ile-Asp(OBu^{t})-Asp(OBu^{t})-PS resin using essentially the same chemical protocols as described for the ACT396 assembly of Example 3. The resultant peptide resin was acetylated by two treatments for 5 min followed by a third treatment for 20 min with a solution of acetic anhydride (5.45 ml) in DMF (54.5 ml). The Ac-Leu-Leu-Leu-Thr(Bu^{t})-Pro-Thr(Bu^{t})-Ala-Lys(Boc)-Ala-Ala-Ser(Bu^{t})-Lys(Boc)-Ile-Asp(OBu^{t})-Asp(OBu^{t})-PS resin was washed with DMF, methanol and ether and dried to produce 59.2 mg of peptide resin. Crude E5 was produced using a mixture of TFA, TIS and water as described for Example 3.

A centrifuged extract of the crude peptide in aqueous acetic acid was purified, in two portions, similarly to that described for Example 3 but using the gradient set at 15% B (time=0 min), 15% B (10 min), 55% B (73 min), 95% (83 min) (gradient name 15S55P), with the product eluting at about 37.5 min. Appropriate fractions were combined and lyophilised to give 11 mg of purified E5. On analytical hplc the purified product eluted at about 22 min with the gradient 5N95D and 11 min with the gradient 30S55D, as described for Example 1. MS (ES) m/e 1598.7 [M+1]⁺. Amino acid analysis, acceptable ratios.

The following compounds were prepared analogously

### Example 6

Decanoyl-Leu-Thr-Pro-Thr-Ala-Arg-Ala-Ala-Ser-Lys-Ile-Asp-Asp-OH (E6)

### Example 7

Decanoyl-Leu-Thr-Pro-Thr-Ala-Asp-Ala-Ala-Ser-Lys-Ile-Asp-Asp-OH (E7)

### Example 8

Decanoyl-Leu-Thr-Pro-Thr-Ala-Asn-Ala-Ala-Ser-Lys-Ile-Asp-Asp-OH (E8)

### Example 9

Decanoyl-Leu-Thr-Pro-Thr-Ala-Leu-Ala-Ala-Ser-Lys-Ile-Asp-Asp-OH (E9)

### Example 10

Hexanoyl-Leu-Thr-Pro-Thr-Ala-Lys-Ala-Ala-Ser-Lys-Ile-Asp-Asp-OH (E10)

The purified peptide was found to elute at 9.6 min with a gradient of 5 to 95% B in 20 min, and at 13.35 min with a gradient of 15 to 35% B in 20 min (Vydac 218TP, 4.6×250 mm, 1.5 ml/min) in which in this case A was 0.1% TFA in water and B was 0.1% TFA in acetonitrile. MS (FAB) 1428.7 [M+1]⁺.

### Example 11

Octanoyl-Leu-Thr-Pro-Thr-Ala-Lys-Ala-Ala-Ser-Lys-Ile-Asp-Asp-OH (E11)

The purified peptide was found to elute at 10.64 min with a gradient of 5 to 95% B in 20 min, and at 8.57 min with a gradient of 25 to 45% B in 20 min under the analytical conditions described for Example 10. MS (FAB) 1456.8 [M+1]⁺. The following compounds may be prepared by an analogous procedure to that given in Example 3 above.

### Example 12

Ac-Leu-Leu-Leu-Thr-Asn-Thr-Ala-Lys-Ala-Ala-Ser-Lys-Lys-Asp-Asp-OH (E12)

Prepared from the intermediate Fmoc-Leu-Thr(Bu^{t})-Asn(Trt)-Thr(Bu^{t})-Ala-Lys(Boc)-Ala-Ala-Ser(Bu^{t})-Lys(Boc)-Lys(Boc)-Asp(OBu^{t})-Asp(OBu^{t})-PS resin.

### Example 13

Ac-Leu-Leu-Leu-Thr-Asn-Thr-Ala-Lys-Ala-Ala-Ser-Ser-Ile-Asp-Asp-OH (E13)

Prepared from the intermediate Fmoc-Leu-Thr(Bu^{t})-Asn(Trt)-Thr(Bu^{t})-Ala-Lys(Boc)-Ala-Ala-Ser(Bu^{t})-Ser(Bu^{t})-Ile-Lys(Boc)-Asp(OBu^{t})-Asp(OBu^{t})-PS resin.

### Example 14

Ac-Leu-Leu-Leu-Thr-Asn-Thr-Ala-Lys-Ala-Glu-Ser-Lys-Ile-Asp-Asp-OH (E14)

Prepared from the intermediate Fmoc-Leu-Thr(Bu^{t})-Asn(Trt)-Thr(Bu^{t})-Ala-Lys(Boc)-Ala-Glu(OBu^{t})-Ser(Bu^{t})-Lys(Boc)-Ile-Asp(OBu^{t})-Asp(OBu^{t})-PS resin.

### Example 15

Ac-Leu-Leu-Leu-Thr-Asn-Asn-Ala-Lys-Ala-Ala-Ser-Lys-Ile-Asp-Asp-OH (E15)

Prepared from the intermediate Fmoc-Leu-Thr(Bu^{t})-Asn(Trt)-Asn(Trt)-Ala-Lys(Boc)-Ala-Ala-Ser(Bu^{t})-Lys(Boc)-Ile-Asp(OBu^{t})-Asp(OBu^{t})-PS resin.

### Example 16

Decanoyl-Leu-Thr-Asn-Thr-Ala-Lys-Ala-Ala-Ser-Ser-Ile-Asp-Asp-OH (E16)

Prepared from the intermediate Fmoc-Leu-Thr(Bu^{t})-Asn(Trt)-Thr(Bu^{t})-Ala-Lys(Boc)-Ala-Ala-Ser(Bu^{t})-Ser(Bu^{t})-Ile-Asp(OBu^{t})-Asp(OBu^{t})-PS resin.

### Example 17

Decanoyl-Leu-Thr-Asn-Asn-Ala-Lys-Ala-Ala-Ser-Lys-Ile-Asp-Asp-OH (E17)

Prepared from the intermediate Fmoc-Leu-Thr(Bu^{t})-Asn(Trt)-Asn(Trt)-Ala-Lys(Boc)-Ala-Ala-Ser(Bu^{t})-Lys(Boc)-Ile-Asp(OBu^{t})-Asp(OBu^{t})-PS resin.

### Example 18

Decanoyl-Leu-Thr-Asn-Thr-Ala-Lys-Ala-Ala-Ser-Lys-Lys-Asp-Asp-OH (E18)

Prepared from the intermediate Fmoc-Leu-Thr(Bu^{t})-Asn(Trt)-Thr(Bu^{t})-Ala-Lys(Boc)-Ala-Ala-Ser(Bu^{t})-Lys(Boc)-Lys(Boc)-Asp(OBu^{t})-Asp(OBu^{t})-PS resin.

### Example 19

Decanoyl-Lys(Flu)-Thr-Pro-Thr-Ala-Lys-Ala-Ala-Ser-Lys-Lys(Bio-X)-Asp-Asp-OH (E19)

Tide compound was assembled by batch process chemistry on an ABI 430A Peptide Synthesiser starting with 0.33 g, 0.25 mmol, of Fmoc-Asp(OBu^{t})-O-Wang resin (Novabiochem, 0.76 mmole/g). The coupling cycle consisted of the following stages: (1) NMP wash; (2) 20% piperidine in NMP, one treatment for 3 min then two treatments for 15 min each; (3) NMP, 5 washes; (4) coupling for 1 hr with the appropriate activated Fmoc-amino acid derivative; (5) NMP, 8 washes; (6) double coupling for 1 hr by repeating step (4); (7) NMP, 7 washes. The activated amino acid derivatives were derived by treating the appropriate Fmoc-amino acid derivative (1 mmole) with 1 equivalent each of HBTU and 1-hydroxybenzotriazole (HOBt) and with 2 equivalents of diisopropylethylamine (DIPEA) in NMP. After eight coupling cycles, the intermediate peptide resin, Fmoc-Ala-Lys(Boc)-Ala-Ala-Ser(Bu^{t})-Lys(Boc)-Lys(Aloc)-Asp(OBu^{t})-Asp(OBu^{t})-O-Wang resin(0.61211 g) was produced. The assembly was continued on 0.3061 g of the latter resin to give, after four further coupling cycles, Fmoc-Lys(Dde)-Thr(Bu^{t})-Pro-Thr(Bu^{t})-Ala-Lys(Boc)-Ala-Ala-Ser(Bu^{t})-Lys(Boc)-Lys(Aloc)-Asp(OBu^{t})-Asp(OBu^{t})-O Wang resin (0.3774 g). This was transferred to a manual peptide synthesiser operating on the bubbler principle, and the Fmoc protection was removed using the protocol, (1) DMF, 5 washes, each 1 min; (2) two treatments with 20% piperidine in DMF for 5 min followed by a third treatment for 10 min; (3) DMF, 6 washes. The resultant H-Lys(Dde)-Thr(Bu^{t})-Pro-Thr(Bu^{t})-Ala-Lys(Boc)-Ala-Ala-Ser(Bu^{t})-Lys(Boc)-Lys(Aloc)-Asp(OBu^{t})-Asp(OBu^{t})-O Wang resin was acylated for 30 min with a solution of decanoic anhydride (0.5 g, 1.5 mmole) in a small amount of DMF to give, after washing with DMF, five times, methanol, three times, ether, two times and then drying, decanoyl-Lys(Dde)-Thr(Bu^{t})-Pro-Thr(Bu^{t})-Ala-Lys(Boc)-Ala-Ala-Ser(Bu^{t})-Lys(Boc)-Lys(Aloc)-Asp(OBu^{t})-Asp(OBu^{t})-O Wang resin. A portion (28.2 mg, ca. 9.4 umole) was vortexed in an argon flushed vial with 0.9 ml of a solution of tetrakis-triphenylphosphine Pd(0) (Lancaster, 99%, 0.2 g, 0.1731 mmole) in 5 ml of an argon flushed solution of anhydrous chloroform (92.5 ml), glacial acetic acid (5 ml) and N-methylmorpholine (2.5 ml). After 2 hr a further 0.9 ml of fresh Pd(0) solution was added and vortexing continued for another 2 hr. The reaction contents were transferred to a manual bubbler and extensively washed as follows: (1) a solution of anhydrous chloroform (92.5 ml), glacial acetic acid (5 ml) and N-methylmorpholine (2.5 ml), 4-times; (2) DMF, 6-times; (3) a solution of diethyldithiocarbamate (0.5 g) and DIPEA (0.5 ml) in DMF (199 ml), 4-times; (4) DMF, 6-times; (5) methanol, 4-times; (5) HOBt in DMF, 2-times; (6) DMF, 3-times; (7) methanol, 3-times; (8) ether, 3-times. The dried peptide resin, decanoyl-Lys(Dde)-Thr(Bu^{t})-Pro-Thr(Bu^{t})-Ala-Lys(Boc)-Ala-Ala-Ser(Bu^{t})-Lys(Boc)-Lys-Asp(OBu^{t})-Asp(OBu^{t})-O Wang resin, was then mixed (argon bubbling) with a solution of 6-((biotinoyl)amino)hexanoic acid, succinimidyl ester (Molecular Probes, biotin-X, SE, 11 mg, 0.0242 mmole, ca. 2.5 equiv) in 1 ml of DMF, followed by the addition of 15.5 ul of a solution of DIPEA (0.174 ml) in DMF (0.826 ml). After mixing for 3 hr and washing the peptide resin with DMF, methanol and ether, the Kaiser ninhydrin test indicated that reaction was not complete. Consequently the above acylation with Biotin-X, SE, was repeated but this time the activated ester was dissolved in 0.5 ml of DMF. Following work-up as before the ninhydrin test was negative and the resulting decanoyl-Lys(Dde)-Thr(Bu^{t})-Pro-Thr(Bu^{t})-Ala-Lys(Boc)-Ala-Ala-Ser(Bu^{t})-Lys(Boc)-Lys(Bio-X)-Asp(OBu^{t})-Asp(OBu^{t})-O Wang resin was dried.

A small aliquot (1 to 3 mg) was treated with 3 ml of 90% TFA (9.5 ml TFA and 0.5 ml water) for 2 hr with occational swirling. The crude decanoyl-Lys(Dde)-Thr-Pro-Thr-Ala-Lys-Ala-Ala-Ser-Lys-Lys(Bio-X)-Asp-Asp-OH was isolated by filtration of the reaction mixture and rinsing the residual resin two-times with fresh TFA, followed by the removal of volatile components from the combined filtrates under vacuum and washing the residual product with ether. An LC-MS indicated the presence of a major peak having the expected molecular weight of 2018.

The fluorescein residue was incorporated into the remaining decanoyl-Lys(Dde)-Thr(Bu^{t})-Pro-Thr(Bu^{t})-Ala-Lys(Boc)-Ala-Ala-Ser(Bu^{t})-Lys(Boc)-Lys(Bio-X)-Asp(OBu^{t})-Asp(OBu^{t})-O Wang resin using the following protocol and the progress of the chemistry was monitored by the ninhydrin test. (1) DMF, 4 washes; (2) removal of the Dde protection by 3 treatments for 3 min with a 2% solution of hydrazine hydrate in DMF; (3) DMF, 6 washes; (4) methanol, 4 washes; (5) DMF, 6 washes; (6) addition of a solution of 5-(and-6)-carboxyfluorescein, succinimidyl ester 5(6)-FAM, SE (Molecular Probes, (5(6)-FAM, SE mixed isomers, 11.83 mg, 0.025 mmole, ca. 2.5 equiv) in 0.5 ml DMF; (7) addition of 25.8 ul of a solution of DIPEA in DMF; (8) mixing for 2 hr; (9) DMF, 2 washes; (10) double coupling by repeating steps (6) to (8); (11) DMF, 6-washes; (12) methanol, 4 washes; (13) ether, 2 washes. The resultant decanoyl-Lys(Flu)-Thr(Bu^{t})-Pro-Thr(Bu^{t})-Ala-Lys(Boc)-Ala-Ala-Ser(Bu^{t})-Lys(Boc)-Lys(Bio-X)-Asp(OBu^{t})-Asp(OBu^{t})-O Wang resin was transferred to a 25 ml round bottomed flask and dried. Treatment of the latter peptide resin with 6 ml of 95% TFA for 2 hr with occational swirling gave crude E19 after isolation by filtration of the reaction mixture and rinsing the residual resin two-times with fresh TFA followed by removal of volatile components from the filtrate under vacuum. Crude E19 gave a major peak on analytical hplc (Hypersil BDS C8, 4.6×250 mm, 1 ml/min, detection at 220 and 256 nm), eluting at about 26 min with the gradient set at 35% B (0 min), 35% B (6 min), 55% B (66 min) (gradient 35H55). Crude E19 was dissolved in aqueous acetic acid and purified in two portions by hplc (Vydac 208TP510, 10×250 mm, 4 ml/min, detection at 220 and 256 nm) with a gradient set at 35% B (time=0 min), 35% B (7 min), 55% B (67 min), (gradient name 35PH55), where A was water, acetonitrile, TFA (1978, 20, 2) and B was acetonitrile, water, TFA (90, 10, 0.1). E19 eluted at about 15 min and relevant fractions were combined, evaporated to dryness under vacuum, re-evaporated from methanol two times and then dried under vacuum. Analysis by LC-MS indicated a purity >90% and confirmed the molecular weight as 2211.

### Example 20

Decanoyl-Leu-Thr-Pro-Lys(Bio-X)-Ala-Lys-Ala-Ala-Ser-Lys-Lys(Flu)-Asp-Asp-OH (E20)

The remaining peptide resin intermediate from the synthesis of Example 19, Fmoc-Ala-Lys(Boc)-Ala-Ala-Ser(Bu^{t})-Lys(Boc)-Lys(Aloc)-Asp(OBu^{t})-Asp(OBu^{t})-O-Wang resin (about 0.3061 g) was further elongated with the ABI 430A to produce Fmoc-Leu-Thr(Bu^{t})-Pro-Lys(Dde)-Ala-Lys(Boc)-Ala-Ala-Ser(Bu^{t})-Lys(Boc)-Lys(Aloc)-Asp(OBu^{t})-Asp(OBu^{t})-O Wang resin (0.3765 g). This was manually converted to decanoyl-Leu-Thr(Bu^{t})-Pro-Lys(Dde)-Ala-Lys(Boc)-Ala-Ala-Ser(Bu^{t})-Lys(Boc)-Lys(Aloc)-Asp(OBu^{t})-Asp(OBu^{t})-O Wang resin, as described for Example 19. The remaining assembly of E20 utilised the same general methodology as was described for Example 19, but applied in a different order. Consequently, the latter peptide resin (29 mg) was treated with Pd(0) for removal of the Aloc protecting group to produce decanoyl-Leu-Thr(Bu^{t})-Pro-Lys(Dde)-Ala-Lys(Boc)-Ala-Ala-Ser(Bu^{t})-Lys(Boc)-Lys-Asp(OBu^{t})-Asp(OBu^{t})-O Wang resin and this was acylated with 5(6)-FAM, SE mixed isomers to produce decanoyl-Leu-Thr(Bu^{t})-Pro-Lys(Dde)-Ala-Lys(Boc)-Ala-Ala-Ser(Bu^{t})-Lys(Boc)-Lys(Flu)-Asp(OBu^{t})-Asp(OBu^{t})-O Wang resin. Reaction of the latter with 2% hydrazine hydrate in DMF removed the Dde protection to give decanoyl-Leu-Thr(Bu^{t})-Pro-Lys-Ala-Lys(Boc)-Ala-Ala-Ser(Bu^{t})-Lys(Boc)-Lys(Flu)-Asp(OBu^{t})-Asp(OBu^{t})-O Wang resin, which was then acylated with biotin-X, SE, to give decanoyl-Leu-Thr(Bu^{t})-Pro-Lys(Bio-X)-Ala-Lys(Boc)-Ala-Ala-Ser(Bu^{t})-Lys(Boc)-Lys(Flu)-Asp(OBu^{t})-Asp(OBu^{t})-O Wang resin. The completed peptide resin was cleaved and deblocked with 6 ml of 95% TFA as described for Example 19 to produce crude E20. Crude E20 gave a major peak eluting at about 32 min on analytical hplc on Hypersil BDS C8 as described for Example 19 but with the gradient set at 35% B (0 min), 35% B (6 min), 65% B (66 min) (gradient 35H65). Crude E20 was dissolved in aqueous acetic acid and purified in two portions by hplc (Vydac 208TP510), as described for Example 19 with the gradient 35PH55. E20 eluted at about 22.5 min and was isolated as described for Example 19. Analysis by LC-MS indicated a purity >90% and confirmed the molecular weight as 2223.

### Example 21

Decanoyl-Lys(Bio-X)-Thr-Pro-Thr-Ala-Lys-Ala-Ala-Ser-Lys-Ile-Asp-Asp-Gly-Lys(Flu)-Asp-OH (E21)

E21 was synthesised analagously to Examples 19 and 20. Fmoc-Asp(OBu^{t})-O-Wang resin (Novabiochem, 0.76 mmole/g, 0.3335 g, 0.25346 mmole) was elongated to Fmoc-Ala-Lys(Boc)-Ala-Ala-Ser(Bu^{t})-Lys(Boc)-Ile-Asp(OBu^{t})-Asp(OBu^{t})-Gly- Lys(Aloc)-Asp(OBu^{t})-O-Wang resin (0.72866 g), as described for Example 1. A portion (0.35132 g) was given four more coupling cycles to produce Fmoc-Lys(Dde)-Thr(Bu^{t})-Pro-Thr(Bu^{t})-Ala-Lys(Boc)-Ala-Ala-Ser(Bu^{t})-Lys(Boc)-Ile-Asp(OBu^{t})-Asp(OBu^{t})-Gly-Lys(Aloc)-Asp(OBu^{t})-O-Wang resin (0.4215g) and this resin was manually converted to decanoyl-Lys(Dde)-Thr(Bu^{t})-Pro-Thr(Bu^{t})-Ala-Lys(Boc)-Ala-Ala-Ser(Bu^{t})-Lys(Boc)-Ile-Asp(OBu^{t})-Asp(OBu^{t})-Gly-Lys(Aloc)-Asp(OBu^{t})-O-Wang resin. A portion (28.1 mg) was treated with Pd(0) to give decanoyl-Lys(Dde)-Thr(Bu^{t})-Pro-Thr(Bu^{t})-Ala-Lys(Boc)-Ala-Ala-Ser(Bu^{t})-Lys(Boc)-Ile-Asp(OBu^{t})-Asp(OBu^{t})-Gly-Lys-Asp(OBu^{t})-O-Wang resin and this was converted to decanoyl-Lys(Dde)-Thr(Bu^{t})-Pro-Thr(Bu^{t})-Ala-Lys(Boc)-Ala-Ala-Ser(Bu^{t})-Lys(Boc)-Ile-Asp(OBu^{t})-Asp(OBu^{t})-Gly-Lys(Flu)-Asp(OBu^{t})-O-Wang resin by reaction with 5(6)-FAM, SE mixed isomers. Further reaction with 2% hydrazine hydrate in DMF gave decanoyl-Lys-Thr(Bu^{t})-Pro-Thr(Bu^{t})-Ala-Lys(Boc)-Ala-Ala-Ser(Bu^{t})-Lys(Boc)-Ile-Asp(OBut)-Asp(OBu^{t})-Gly-Lys(Flu)-Asp(OBu^{t})-O-Wang resin and acylation of this with Biotin-X, SE, gave decanoyl-Lys(Bio-X)-Thr(Bu^{t})-Pro-Thr(Bu^{t})-Ala-Lys(Boc)-Ala-Ala-Ser(Bu^{t})-Lys(Boc)-Ile-Asp(OBu^{t})-Asp(OBu^{t})-Gly-Lys(Flu)-Asp(OBu^{t})-O-Wang resin. This was cleaved and deblocked with 95% TFA as for Example 20 to give crude E21, which displayed a major peak eluting at 22.5 min with the gradient 35H65 as described for Example 20. The crude E21 was purified by hplc as given for Example 20 with the gradient 35PH55 and eluted at about 13 min. Analysis by LC-MS indicated a purity >90% and confirmed the molecular weight as 2495.

### Example 22

Decanoyl-Leu-Thr-Pro-Lys(Bio-X)-Ala-Lys-Ala-Ala-Ser-Lys-Ile-Asp-Asp-Gly-Lys(Flu)-Asp-OH (E22)

The remaining peptide resin intermediate from the synthesis of Example 21, Fmoc-Ala-Lys(Boc)-Ala-Ala-Ser(Bu^{t})-Lys(Boc)-Ile-Asp(OBu^{t})-Asp(OBu^{t})-Gly-Lys(Aloc)-Asp(OBu^{t})-O-Wang resin (about 0.37734 g) was further elongated with the ABI 430A to produce Fmoc-Leu-Thr(Bu^{t})-Pro-Lys(Dde)-Ala-Lys(Boc)-Ala-Ala-Ser(Bu^{t})-Lys(Boc)-Ile-Asp(OBu^{t})-Asp(OBu^{t})-Gly-Lys(Aloc)-Asp(OBu^{t})-O-Wang resin(0.4267 g) and then was manually converted to decanoyl-Leu-Thr(Bu^{t})-Pro-Lys(Dde)-Ala-Lys(Boc)-Ala-Ala-Ser(Bu^{t})-Lys(Boc)-Ile-Asp(OBu^{t})-Asp(OBu^{t})-Gly-Lys(Aloc)-Asp(OBu^{t})-O-Wang resin, as described for Example 1. A portion (29.6 mg) was treated with Pd(0) to produce decanoyl-Leu-Thr(Bu^{t})-Pro-Lys(Dde)-Ala-Lys(Boc)-Ala-Ala-Ser(Bu^{t})-Lys(Boc)-Ile-Asp(OBu^{t})-Asp(OBu^{t})-Gly-Lys-Asp(OBu^{t})-O-Wang resin, which on acylation with 5(6)-FAM, SE mixed isomers gave decanoyl-Leu-Thr(Bu^{t})-Pro-Lys(Dde)-Ala-Lys(Boc)-Ala-Ala-Ser(Bu^{t})-Lys(Boc)-Ile-Asp(OBu^{t})-Asp(OBu^{t})-Gly-Lys(Flu)-Asp(OBu^{t})-O-Wang resin. Further reaction with 2% hydrazine hydrate in DMF gave decanoyl-Leu-Thr(Bu^{t})-Pro-Lys-Ala-Lys(Boc)-Ala-Ala-Ser(Bu^{t}) Lys(Boc)-Ile-Asp(OBu^{t})-Asp(OBu^{t})-Gly-Lys(Flu)-Asp(OBu^{t})-O-Wang resin, which reacted with Biotin-X, SE, to give decanoyl-Leu-Thr(Bu^{t})-Pro-Lys(Bio-X)-Ala-Lys(Boc)-Ala-Ala-Ser(Bu^{t})-Lys(Boc)-Ile-Asp(OBu^{t})-Asp(OBu^{t})-Gly-Lys(Flu)-Asp(OBu^{t})-O-Wang resin. The latter was cleaved and deblocked with 95% TFA as for Example 20 to give crude E22, which displayed a major peak eluting at about 27 min with the gradient 35H65 as described for Example 20. The crude E22 was purified by hplc as given for Example 20 with the gradient 35PH55 and eluted at about 24.5 min. Analysis by LC-MS indicated a purity >90% and confirmed the molecular weight as 2507.

### Example 23

Decanoyl-Lys(Flu)-Thr-Pro-Thr-Ala-Lys-Ala-Ala-Ser-Lys-Lys(TAMRA)-Asp-Asp-OH (E23)

A portion of the decanoyl-Lys(Dde)-Thr(Bu^{t})-Pro-Thr(Bu^{t})-Ala-Lys(Boc)-Ala-Ala-Ser(Bu^{t})-Lys(Boc)-Lys(Aloc)-Asp(OBut)-Asp(OBu^{t})-O Wang resin from Example 19 (30 mg) was treated, as before, with tetrakis-triphenylphosphine Pd(0) in a solution of anhydrous chloroform, glacial acetic acid and N-methylmorpholine to produce decanoyl-Lys(Dde)-Thr(Bu^{t})-Pro-Thr(Bu^{t})-Ala-Lys(Boc)-Ala-Ala-Ser(Bu^{t})-Lys(Boc)-Lys-Asp(OBu^{t})-Asp(OBu^{t})-O Wang resin. This was reacted with a solution of 5-(and-6-)-carboxytetramethylrhodamine, succinimyl ester (Molecular Probes, mixed isomers, ca. 2.36 equiv) in DMF, under conditions similar to those described for the incorporation of the Flu residue in Example 19, produced decanoyl-Lys(Dde)-Thr(Bu^{t})-Pro-Thr(Bu^{t})-Ala-Lys(Boc)-Ala-Ala-Ser(Bu^{t})-Lys(Boc)-Lys(TAMRA)-Asp(OBu^{t})-Asp(OBu^{t})-O Wang resin.

Treatment of the latter peptide resin with 2% hydrazine hydrate in DMF gave decanoyl-Lys-Thr(Bu^{t})-Pro-Thr(Bu^{t})-Ala-Lys(Boc)-Ala-Ala-Ser(Bu^{t})-Lys(Boc)-Lys(TAMRA)-Asp(OBu^{t})-Asp(OBu^{t})-O Wang resin, which was acylated, as previously described, with 5(6)-Fam, SE mixed isomers, to produce decanoyl-Lys(Flu)-Thr(Bu^{t})-Pro-Thr(Bu^{t})-Ala-Lys(Boc)-Ala-Ala-Ser(Bu^{t})-Lys(Boc)-Lys(TAMRA)-Asp(OBu^{t})-Asp(OBu^{t})-O Wang resin.

Treatment of the latter peptide resin with 95% TFA, as previously described for Example 19, gave crude E23, that showed two major peaks eluting at about 29 and 31 min, respectively, on analytical hplc with the gradient 35H65.

Both major peaks were isolated by prep-hplc, as previously described, with the gradient 35PH55, eluting at about 16 (23A) and 20 (23C) mins, respectively. Since each product displayed the expected MW of 2284, each product consists of unresolved isomers.

### Example 24

Decanoyl-Lys(TAMRA)-Thr-Pro-Thr-Ala-Lys-Ala-Ala-Ser-Lys-Lys(Flu)-Asp-Asp-OH (E24)

E24 was synthesised in an analogous way to Example 23, starting with 31 mg of the peptide resin intermediate, decanoyl-Lys(Dde)-Thr(Bu^{t})-Pro-Thr(Bu^{t})-Ala-Lys(Boc)-Ala-Ala-Ser(Bu^{t})-Lys(Boc)-Lys(Aloc)-Asp(OBu^{t})-Asp(OBu^{t})-O Wang resin from Example 19. The Flu residue was first incorporated and then the TAMRA residue. Crude E24 showed a similar analytical hplc profile as E23 with gradient 35H65. The two major products were isolated by prep-hplc with the gradient set at 30% B (time=0 min), 30% B (7 min), 55% B (67 min), (gradient name 30PH55), eluting at about 29 (24C) and 33 (24D) min, respectively, and each displayed a MW of 2284.

### Example 25

Decanoyl-Leu-Thr-Pro-Lys(Flu)-Ala-Lys-Ala-Ala-Ser-Lys-Lys(TAMRA)-Asp-Asp-OH (E25)

E25 was synthesised using the same sequence of operations as described for Example 23, starting from 35 mg of the peptide resin intermediate, decanoyl-Leu-Thr(Bu^{t})-Pro-Lys(Dde)-Ala-Lys(Boc)-Ala-Ala-Ser(Bu^{t})-Lys(Boc)-Lys(Aloc)-Asp(OBu^{t})-Asp(OBu^{t})-O Wang resin, from Example 20. The two major peaks were isolated by prep-hplc, as previously described, with the gradient 35PH55, eluting at about 29 (25B) and 33 (25C) mins, respectively, and displayed a MW of 2296.

### Example 26

Decanoyl-Leu-Thr-Pro-Lys(Flu)-Ala-Lys-Ala-Ala-Ser-Lys-Lys(BioX)Asp-Asp-OH (E27)

E26 was synthesised by methods generally described herein.

### Example 27

Decanoyl-Leu-Thr-Pro-Thr-Ala-Tyr-Ala-Ala-Ser-Lys-Ile-Asp-Asp-OH (E27)

E27 was synthesised in an analogous manner to that of Example 4.

The compounds of the following Examples 28 to 39 were prepared by methods generally described herein:

### Example 28

Decanoyl-Leu-Thr-Pro-Thr-Ala-Arg-Ala-Ala Asp-Gly-Pro-Arg-Ser-OH (E28)

### Example 29

Decanoyl-Leu-Thr-Pro-Thr-Ala-Arg-Ala-Ala-Asp-Glu-Pro-Arg-Ser-OH (E29)

### Example 30

Decanoyl-Leu-Thr-Pro-Thr-Ala-Arg-Ala-Ala-Asp-Leu-Pro-Arg-Ser-OH (E30)

### Example 31

Decanoyl-Leu-Thr-Pro-Thr-Ala-Arg-Ala-Ala-Asp-Pro-Asp-Ser-Arg-OH (E31)

### Example 32

Decanoyl-Leu-Thr-Pro-Thr-Ala-Arg-Ala-Ala-Pro-Gly-Asp-Arg-Ser-OH (E32)

### Example 33

Decanoyl-Leu-Thr-Pro-Thr-Ala-Arg-Ala-Ala-Pro-Ala-Thr-Glu-Glu-OH (E33)

### Example 34

Decanoyl-Leu-Ser-Leu-Pro-Ala-His-Ala-Ala-Asp-Gly-Pro-Arg-Ser-OH (E34)

### Example 35

Decanoyl-Leu-Ser-Leu-Pro-Ala-His-Ala-Ala-Asp-Glu-Pro-Arg-Ser-OH (E35)

### Example 36

Decanoyl-Leu-Ser-Leu-Pro-Ala-His-Ala-Ala-Asp-Leu-Pro-Arg-Ser-OH (E36)

### Example 37

Decanoyl-Leu-Ser-Leu-Pro-Ala-His-Ala-Ala-Asp-Pro-Asp-Ser-Arg-OH (E37)

### Example 38

Decanoyl-Leu-Ser-Leu-Pro-Ala-His-Ala-Ala-Pro-Gly-Asp-Arg-Ser-OH (E38)

### Example 39

Decanoyl-Leu-Ser-Leu-Pro-Ala-His-Ala-Ala-Pro-Ala-Thr-Glu-Glu-OH (E39)

The compounds of the following Examples 40 to 45 are prepared by methods generally described herein:

### Example 40

Decanoyl-Lys(Flu)-Ser-Leu-Pro-Ala-His-Ala-Ala-Lys(Tamra)-Leu-Pro-Arg-Ser-OH

### Example 41

Decanoyl-Lys(Tamra)-Ser-Leu-Pro-Ala-His-Ala-Ala-Lys(Flu)-Leu-Pro-Arg-Ser-OH

### Example 42

Decanoyl-Lys(Flu)-Ser-Leu-Pro-Ala-His-Ala-Ala-Asp-Lys(TAMRA)-Pro-Arg-Ser-OH

### Example 43

Decanoyl-Lys(TAMRA)-Ser-Leu-Pro-Ala-His-Ala-Ala-Asp-Lys(Flu)-Pro-Arg-Ser-OH

### Example 44

Decanoyl-Lys(Flu)-Ser-Leu-Pro-Ala-His-Ala-Ala-Asp-Leu-Pro-Lys(TAMRA)-Ser-OH

### Example 45

Decanoyl-Lys(TAMRA)-Ser-Leu-Pro-Ala-His-Ala-Ala-Asp-Leu-Pro-Lys(Flu)-Ser-OH

Abbreviations:

Trt trityl

Boc butoxycarbonyl

Fmoc 9-fluorenylmethoxycarbonyl

HBTU 2-(1H-benzotriazole-1-yl)-1,1,3,3-tetramethyluronium hexafluorophosphate

TBTU 2-(1H-benzotriazole-1-yl)-1,1,3,3-tetramethyluronium tetrafluoroborate

DMF dimethylformamide

TFA trifluoroacetic acid

Aloc allyloxycarbonyl

Dde 1-(4,4-dimethyl-2,6-dioxocyclohex-1-ylidene)ethyl

NMP N-methylpyrolidine

Bu^{t} t-Butyl

TFA trifluoroacetic acid

Flu fluorescein-5/6-carbonyl

Bio-X 6-((biotinoyl)amino)hexanoyl

TAMRA tetramethylrhodamine-5/6-carbonyl.

ELISA enzyme linked immunosorbent assay

AMPSO 3-[1,1-Dimethyl-2-hydroxyethyl)amino]-2-hydroxypropanesulfonic acid

EDTA ethylenediaminetetraacetic acid

Triton X t-octylphenoxypolyethoxyethanol

Substrate Assays

1. HPLC/MS Assay Protocol

SpsB (Cregg K. M and Black M. T. J. Bacteriol. 1996, 5712) (1 μM) was reacted at a 1:500 ratio with test substrate (0.5 mM) in an assay buffer consisting of 50 mM 2-[N-Cyclohexylamino]ethanesulfonic acid (CHES, Sigma-Aldrich) adjusted to pH 8.5, 1 mM ethylenediaminetetraacetic acid, disodium dihydrate (EDTA, Pierce & Warriner), 1 mM phenylmethylsulfonyl fluoride (PMSF, Sigma-Aldrich) and 0.1% reduced Triton-X100 (Sigma-Aldrich). Test substrates were dissolved in dimethyl sulphoxide (DMSO) and then diluted such that the final solvent consisted of 20% DMSO, 1% reduced Triton-X100 and 79% water. For the assay 10 μL of substrate solution was added to 2 μL SpsB, 2 μL of a 10 fold concentrated assay buffer and 6 μL of water. The reaction was incubated at 37° C. and 5 μL aliquots were then removed at suitable time points. Each aliquot was acidified with TFA to prevent further reaction and analysed by high performance liquid chromatography electrospray ionisation mass spectrometry (HPLC ESI/MS). The HPLC system consisted of a Brownlee Aquapore RP300 7 μM 100×2.1 mm column maintained at 40° C. The eluents used were A, 0.1% trifluoroacetic acid (TFA, Sigma-Aldrich) in water (HPLC grade, Fischer Scientific) and B, 0.1% TFA in acetonitrile (190 HPLC grade, Romil). A gradient system was employed starting at 5% B rising to 75% B over 14 minutes, which was maintained for a further 5 minutes, with a 10 minute post run column equilibration at 5% B. The flow rate was 200 μL per minute and UV detection was at 214 nm. After the UV all the eluent was directed into the electrospray atmospheric pressure ionisation source of the LCQ (ThermoQuest) quadrupole ion trap mass spectrometer. Spectra were acquired over the mass to charge ratio of 150 to 2000 and processed using ThermoQuest Navigator software.

The mass spectra corresponding to the peaks observed in the UV chromatogram were selected and used to identify the full length and N-terminal cleavage product of each peptide substrate. The areas of these peaks, usually from the UV chromatogram although ion intensity chromatograms from the mass spectrometer response were used in cases of poor resolution between the substrate and cleavage product, were used to determine the approximate cleavage rate from the percentage N-terminal product produced. Some peptide substrates were partially insoluble in the required buffer system and the figure calculated for these was an approximation based on the observed ratios from the supernatant after incubation.

A similar protocol was used to assay the test substrates against E. coli leader peptidase in which the N terminus of LP1 has been mutated to remove the internal cleavage site (Ala-38 to Tyr and Ala-40 to Thr).

Results

Examples E4, E6, E8, E11, E28 and E33 exhibited >80% processing after 30 minutes with S. aureus SpsB.

Examples E4, E6 and E9 exhibited >/=40% processing after 5 minutes, and E27, E28 and E34 to E39>10% after 2 minutes, with E. coli leader peptidase.

2. S. aureus SpsB Signal Peptidase Fluorescence Quench (FQ) Assay

Peptide test substrate* (38-109 nM) was incubated with S. aureus SpsB (Cregg K. M and Black M. T., J. Bacteriol. 1996, 5712) (40 nM) at room temperature in buffer (50 mM AMPSO, 1 mM EDTA, 0.1% Triton X-100) in a Dynex black 96 well U-bottomed microtitre plate. Cleavage of the peptide was monotored using a BMG Polarstar fluorescence plate reader fitted with a 485 nm excitation filter and a 520-35 nm emission filter.

*Concentrations of test substrate used: E23A=109 nM, E23C=40 nM, E24C=73 nM, E24D=38 nM, E25B=87 nM, E25C=39 nM.

Results

Examples E23C, E24C, E24D displayed catalytic efficiencies kcat/Km >/=5000 M⁻¹ sec^{-1.}

Examples E24C, E24D produced signal changes >10-fold.

3. S. aureus spsB Signal Peptidase Fluorescence Polarisation (FP) Assay

1.0 μM test substrate was incubated with S. aureus SpsB (Cregg K. M and Black M. T., J. Bacteriol. 1996, 5712) (40 nM) for 1 hour in 50 mM AMPSO pH 8.5, 1 mM EDTA, 0.1% Triton X-100 at room temperature. The assay was quenched and the FP signal developed by diluting assay aliquots 10-fold in 2μM avidin. The stopped assays were measured on a BMG Polarstar fluorescence polarisation platereader calibrated to provide 90% full scale deflection on both photomultiplier tubes and an FP value of 25 mP with 1 μM fluorescein.

[Reference: Leane M. Levine, Marshall L. Michener, Mihaly V. Toth and Barry C. Holwerda (1997) Measurement of specific protease activity utilizing fluorescence polarisation. Anal. Biochem. 247, 83-88].

Results

Examples 19-22 were tested and displayed catalytic efficiencies kcat/Km >/=8000 M⁻¹ sec⁻¹ and signal changes >/=150 mP

4. Signal Peptidase Fluorescence Correlation Spectroscopy (FCS) Assay

S. aureus SpsB (Cregg K. M and Black M. T., J. Bacteriol. 1996, 5712) (80 nM) was incubated with test substrate (1 μM) in 50 mM AMPSO, pH 8.5, 0.1% Triton X-100, 1 mM EDTA. The reaction was quenched at 4 minute intervals by diluting the assay components 100-fold (to 10 nM of test substrate) into 2 μM avidin, in the same buffer. The stopped samples were then analysed by fluorescence correlation spectroscopy (FCS).

Results

For Example 20 the exponential decay constant described by the decrease in intact peptide equated to a k_{cat/}Kₘ=6800 M⁻¹s⁻¹.

1. A compound of formula (I):
[A][A1][A2][A3]*[A4] (I)
where:
* is the cleavage site
[A] is selected from:
(i) 2 to 12 hydrophobic amino acid residues substituted at the N-terminus by C₁₋₅ alkanoyl or phenylC₁₋₄alkanoyl optionally substituted by C₁₋₄alkyl, C₁₋₄alkoxy or halogen; and
(ii) a hydrophobic acyl residue;
[A1] is 1 to 3 amino acid residues selected from A, F, G, I, L, M, N, S, T, V;
[A2] is 3 amino acid residues selected from those favoured in a beta- or helical turn;
[A3] is 3 amino acid residues X—B-Z, where X is selected from A, G, S, T, and V, B is any amino acid residue and Z is selected from A, G and S; and
[A4] is 2 to 8 amino acid residues chosen predominantly from those that are favoured in beta-turns and enhance water solubility:
and optionally wherein:
one amino acid in one of [A1] and [A2] is replaced by X1 comprising an amino acid bearing a marker group; and one amino acid in [A4] other than the residue immediately adjacent to [A3] is replaced by X2 comprising an amino acid bearing a marker group; such that X1 and X2 form a marker pair;
[A2] residues are selected from G, L, N, P, S and T;
X is selected from A, S and V;
B is selected from D, F, H, I, K, L, N, Q, R, V or Y; and
Z is selected from A and S.

2. A compound according to claim 1 wherein [A] (i) hydrophobic amino acid residues are selected from A, F, I, L, M and V, halogen is chlorine and the N-terminal substituent is acetyl.

3. A compound according to claim 1 or 2 wherein [A1] is 1 amino acid chosen from G, L or N.

4. A compound according to claim 1 wherein [A] (ii) hydrophobic acyl residue is selected from phenylC₁₋₁₂alkanoyl, biphenylC₁₋₁₂alkanoyl, phenoxyphenylC₁₋₁₂alkanoyl Or C₅₋₁₆ alkanoyl, wherein the phenyl moieties are optionally substituted by C₁₋₈alkyl, C₁₋₈alkoxy or halogen.

5. A compound according to claim 4 wherein [A] (ii) is C₁₀ alkanoyl.

6. A compound according to claim 4 or 5 wherein [A1] is 1 or 2 amino acids selected from F, I, L and V, most preferably 1 amino acid from F, I, L and V. [A1] is most preferably L.

7. A compound according to any preceding claim wherein [A2] is 3 amino acid residues selected from A, F, G, I, L, N, P, S, T, V.

8. A compound according to any preceding claim wherein X is A, S or V, B is a neutral or basic amino acid residue and Z is A or S.

9. A compound according to any preceding claim wherein the first amino acid residue in [A4] is A, D, E, Q or S and the remaining amino acid residues are selected from A, D, E, F, G, I, K, L, N, P, Q, R, S, T and V with not more than two of each of G, P and R and no more than 2 amino acid residues being selected from F, I, L and V.

10. A compound according to claim 1 of formula (II):
[A]-a1-a2-a3-a4-a5-a6-a7-a8-a9-a10 (II)
where:
[A] is as defined in formula (I);
a1 is leu or X1;
and either
(i)
a2 is thr or X1;
a3 is pro;
a4 is thr or X1;
a5 is ala-(lys or arg or asn)-ala*-ala-;
a6 is ser or X2;
a7 is lys or X2;
a8 is ile or X2;
a9 is asp or X2; and
a10 is asp or X2;
or
(ii)
a2 is ser or X1;
a3 is leu or X1;
a4 is pro;
a5 is ala-(lys or arg or his)-ala*-ala-;
a6 is asp or X2;
a7 is leu or gly or X2;
a8 is pro;
a9 is arg or X2;
a10 is ser or X2.

11. A compound according to claim 10 wherein in compounds of formula (II)(i) X1 is at a1 or a4 and X2 is at a8 or a10, and in compounds of formula (II)(ii) X1 is at a1 or a3 and X2 is at a6, a7 or a9.

12. A compound according to any preceding claim wherein the X1/X2 marker group pair is a fluorophore pair or a fluorophore/biotin ligand combination.

13 An assay system for testing for bacterial signal peptidase inhibitors which comprises contacting a bacterial signal peptidase and a compound of formula (I) according to claim 1 with a test compound and measuring inhibition of the cleavage of the compound of formula (I) by the peptidase.


SEQUENCE LISTING (64 sequences)

SEQ ID NO: 1 - protein, 22 aa, Artificial Sequence
  unsure (2)(3)(4)(5)(6)(7)(8)(9)(10)(11)(12)(13)(14)(15)(16)(17),(18)(19)(20)(21): Whereby these proteins precede the cleavage site; in a signal sequence containing hydrophobic; membrane spanning helical domain which is followed
  by a more polar region.
AXXXXXXXXXXXXXXXXXXXXA

SEQ ID NO: 2 - protein, 23 aa, Artificial Sequence
  A 23 amino acid peptide comprising the -15 to +8
  residues of the M13 procoat protein.
ASVAVATLVPMLSFAAEGDDPAK

SEQ ID NO: 3 - protein, 17 aa, Artificial Sequence
  Whereby these proteins are related to the 23 amino; acid peptide comprising the -15 to +8 residues of
  the M13 procoat protein
WLVPNLLSFAAEGDDPA

SEQ ID NO: 4 - protein, 9 aa, Artificial Sequence
  Corresponding to the -7 to +2 sequence of maltose
  binding protein.
FSASALAKI

SEQ ID NO: 5 - protein, 5 aa, Artificial Sequence
  The shortest minimum required sequence for; processing which corresponds to the -7 to +2
  maltose binding protein.
ALAKI

SEQ ID NO: 6 - protein, 9 aa, Artificial Sequence
  Related to previous sequence.  The shortest; minimum required sequence for processing which; corresponds to the -7 to +2 maltose binding
  protein.
WSASALAKI

SEQ ID NO: 7 - protein, 9 aa, Artificial Sequence
  Related to previous sequence.  The shortest; minimum required sequence for processing which; corresponds to the -7 to +2 maltose binding
  protein.
WSASALAKI

SEQ ID NO: 8 - protein, 9 aa, Artificial Sequence
  A fluorescent substrate for continuous assay
WSASALAKI

SEQ ID NO: 9 - protein, 6 aa, Artificial Sequence
  The preferred hydrophobic amino acid residues; substituted in the compound which are substrates; for bacterial signal peptidases and which are of; use for configuring assays to detect inhibitors of
  the enzymes.
AFILMV

SEQ ID NO: 10 - protein, 3 aa, Artificial Sequence
  Proteins represent 1 to 3 preferred amino acid; residues substituted in the compound which are; substrates for bacterial signal peptidases and; which are of use for configuring assays to detect
  inhibitors of the enzymes.
GLN

SEQ ID NO: 11 - protein, 4 aa, Artificial Sequence
  In this instance, proteins comprise an amino-terminal
  hyrophobic acyl residue
FILV

SEQ ID NO: 12 - protein, 3 aa, Artificial Sequence
  Proteins represent the preferred 1 to 3 amino acid
  residues.
TPT

SEQ ID NO: 13 - protein, 3 aa, Artificial Sequence
  Proteins represent the preferred 1 to 3 amino acid
  residues.
SLP

SEQ ID NO: 14 - protein, 12 aa, Artificial Sequence
  Proteins represent the preferred 4 to 6 amino acid
  residues
ADEGIKLPRSTV

SEQ ID NO: 15 - protein, 9 aa, Artificial Sequence
  Escherichia coli leader peptidase substrates
PADEGLRST

SEQ ID NO: 16 - protein, 26 aa, Artificial Sequence
  unsure (2)(4)(7)(9)(13)(14)(16)(17)(19)(20)(22)(23)(25)(26): Preferred substrates for Sps B of Staphylococcus
  aureus
LXTXPTXAXAASXXKXXIXXDXXDXX

SEQ ID NO: 17 - protein, 23 aa, Artificial Sequence
  unsure (2)(4)(6)(9)(13)(14)(16)(19)(20)(22)(23): Preferred substrates for Sps B of Staphylococcus
  aureus
LXSXLXPAXAADXXLXPRXXSXX

SEQ ID NO: 18 - DNA, 25 bp, Artificial Sequence
  Escherichia coli leader peptidase
gtcaggcagcggcgcaggcggctcg

SEQ ID NO: 19 - DNA, 25 bp, Artificial Sequence
  Escherichia coli leader peptidase
gtcaggcagcgtatcagacggctcg

SEQ ID NO: 20 - protein, 15 aa, Artificial Sequence
  Proteins assembled by continuous flow chemistry on
  a Millipore 9050 Peptide Synthesizer
LLLTNTAKAASKIDD

SEQ ID NO: 21 - protein, 13 aa, Artificial Sequence
  Proteins assembled by continuous flow chemistry on
  a Millipore 9050 Peptide Synthesizer
LTNTAKAASKIDD

SEQ ID NO: 22 - protein, 13 aa, Artificial Sequence
  Synthesized proteins
LTNTAKAESKIDD

SEQ ID NO: 23 - protein, 13 aa, Artificial Sequence
  The completion and purification of proteins
  synthesized in sequence 23 above
LTPTAKAASKIDD

SEQ ID NO: 24 - protein, 15 aa, Artificial Sequence
  Intermediate peptide resin
LLLTPTAKAASKIDD

SEQ ID NO: 25 - protein, 15 aa, Artificial Sequence
  Intermediate peptide resin
LLLTPTAKAASKIDD

SEQ ID NO: 26 - protein, 13 aa, Artificial Sequence
  Intermediate peptide resin
LTPTADAASKIDD

SEQ ID NO: 27 - protein, 13 aa, Artificial Sequence
  Intermediate peptide resin
LTPTANAASKIDD

SEQ ID NO: 28 - protein, 13 aa, Artificial Sequence
  Intermediate peptide resin
LTPTALAASKIDD

SEQ ID NO: 29 - protein, 13 aa, Artificial Sequence
  Intermediate peptide resin
LTPTAKAASKIDD

SEQ ID NO: 30 - protein, 13 aa, Artificial Sequence
  Intermediate peptide resin
LTPTAKAASKIDD

SEQ ID NO: 31 - protein, 15 aa, Artificial Sequence
  Intermediate peptide resin
LLLTNTAKAASKKDD

SEQ ID NO: 32 - protein, 15 aa, Artificial Sequence
  Intermediate peptide resin
LLLTNTAKAASSIDD

SEQ ID NO: 33 - protein, 15 aa, Artificial Sequence
  Intermediate peptide resin
LLLTNTAKAESKIDD

SEQ ID NO: 34 - protein, 15 aa, Artificial Sequence
  Intermediate peptide resin
LLLTNNAKAASKIDD

SEQ ID NO: 35 - protein, 13 aa, Artificial Sequence
  Intermediate peptide resin
LTNTAKAASSIDD

SEQ ID NO: 36 - protein, 14 aa, Artificial Sequence
  Intermediate peptide resin
LTNNAKAASSKIDD

SEQ ID NO: 37 - protein, 13 aa, Artificial Sequence
  Intermediate peptide resin
LTNTAKAASKKDD

SEQ ID NO: 38 - protein, 13 aa, Artificial Sequence
  Intermediate peptide resin
KTPTAKAASKKDD

SEQ ID NO: 39 - protein, 13 aa, Artificial Sequence
  Intermediate peptide resin
LTPKAKAASKKDD

SEQ ID NO: 40 - protein, 16 aa, Artificial Sequence
  Intermediate peptide resin
KTPTAKAASKIDDGKD

SEQ ID NO: 41 - protein, 13 aa, Artificial Sequence
  Intermediate peptide resin
LTPKAKAASKIDD

SEQ ID NO: 42 - protein, 13 aa, Artificial Sequence
  Intermediate peptide resin
KTPTAKAASKKDD

SEQ ID NO: 43 - protein, 13 aa, Artificial Sequence
  Intermediate peptide resin
KTPTAKAASKKDD

SEQ ID NO: 44 - protein, 13 aa, Artificial Sequence
  Intermediate peptide resin
LTPKAKAASKKDD

SEQ ID NO: 45 - protein, 13 aa, Artificial Sequence
  Intermediate peptide resin
LTPKAKAASKKDD

SEQ ID NO: 46 - protein, 13 aa, Artificial Sequence
  Intermediate peptide resin
LTPTAYAASKIDD

SEQ ID NO: 47 - protein, 13 aa, Artificial Sequence
  Intermediate peptide resin
LTPTARAADGPRS

SEQ ID NO: 48 - protein, 13 aa, Artificial Sequence
  Intermediate peptide resin
LTPTARAADEPRS

SEQ ID NO: 49 - protein, 13 aa, Artificial Sequence
  Intermediate peptide resin
LTPTARAADLPRS

SEQ ID NO: 50 - protein, 13 aa, Artificial Sequence
  Intermediate peptide resin
LTPTARAADPDSR

SEQ ID NO: 51 - protein, 13 aa, Artificial Sequence
  Intermediate peptide resin
LTPTARAAPGDRS

SEQ ID NO: 52 - protein, 13 aa, Artificial Sequence
  Intermediate peptide resin
LTPTARAAPATEE

SEQ ID NO: 53 - protein, 13 aa, Artificial Sequence
  Intermediate peptide resin
LSLPAHAADGPRS

SEQ ID NO: 54 - protein, 13 aa, Artificial Sequence
  Intermediate peptide resin
LSLPAHAADEPRS

SEQ ID NO: 55 - protein, 13 aa, Artificial Sequence
  Intermediate peptide resin
LSLPAHAADLPRS

SEQ ID NO: 56 - protein, 13 aa, Artificial Sequence
  Intermediate peptide resin
LSLPAHAADPDSR

SEQ ID NO: 57 - protein, 13 aa, Artificial Sequence
  Intermediate peptide resin
LSLPAHAAPGDRS

SEQ ID NO: 58 - protein, 13 aa, Artificial Sequence
  Intermediate peptide resin
LSLPAHAAPATEE

SEQ ID NO: 59 - protein, 13 aa, Artificial Sequence
  unsure (1)(9): Intermediate peptide resin
XSLPAHAAXLPRS

SEQ ID NO: 60 - protein, 13 aa, Artificial Sequence
  unsure (1)(9): Intermediate peptide resin
XSLPAHAAXLPRS

SEQ ID NO: 61 - protein, 13 aa, Artificial Sequence
  unsure (1)(10): Intermediate peptide resin
XSLPAHAADXPRS

SEQ ID NO: 62 - protein, 13 aa, Artificial Sequence
  unsure (1)(10): Intermediate peptide resin
XSLPAHAADXPRS

SEQ ID NO: 63 - protein, 13 aa, Artificial Sequence
  unsure (1)(12): Intermediate peptide resin
XSLPAHAADLPXS

SEQ ID NO: 64 - protein, 12 aa, Artificial Sequence
  unsure (1): Intermediate peptide resin
XSLPAHAADLPS